SEQUENCE LISTING (5 sequences)

SEQ ID NO: 1 - DNA, 20 bp, Artificial Sequence
  chemically synthesized
gggattatttttataaggtt

SEQ ID NO: 2 - DNA, 23 bp, Artificial Sequence
  chemically synthesized
tactaaaaactctaaaccccatc

SEQ ID NO: 3 - DNA, 21 bp, Artificial Sequence
  chemically synthesized
ttcgtcgtcgtagttttcgtt

SEQ ID NO: 4 - DNA, 18 bp, Artificial Sequence
  chemically synthesized
tagtgagtacgcgcggtt

SEQ ID NO: 5 - DNA, 25 bp, Artificial Sequence
  chemically synthesized
cccatccccaaaaacacaaaccaca

(19) United States
(12) Patent Application Publication
(10) Pub. No.: US 2007/0160991 A1
(43) Pub. Date: Jul. 12, 2007

(54) Method for the methylation analysis of dna

(75) Inventor: Juergen Distler, Berlin (DE)

Correspondence Address: KRIEGSMAN & KRIEGSMAN, 30 TURNPIKE ROAD, SUITE 9, SOUTHBOROUGH, MA, 01772, US

(73) Assignee: EPIGENOMICS AG, Berlin (DE)

(21) Appl. No.: 10/573,939

(22) PCT Filed: Sep. 27, 2004
(86) PCT No.: PCT/DE04/02178

§ 371 Date: Oct. 16, 2006

(30) Foreign Application Priority Data
Sep. 30, 2003 (DE) 103 46 363.1

(51) Int. Cl. C12Q 1/68; C12P 19/34

(52) U.S. Cl. 435/6; 435/91.2

(57) ABSTRACT

The present invention concerns a method for the detection of methylated DNA against a background of unmethylated DNA. The double strands of the DNA to be investigated are separated and then reassociated with the formation of hemimethylated double strands. After this, the hemimethylated DNA is enzymatically converted into completely methylated DNA. The quantity of methylated DNA is thus increased. Then the methylated DNA can be analyzed by various methods. The method according to the invention is suitable particularly for the diagnosis and prognosis of cancer disorders and other diseases associated with a change in the methylation status as well as for predicting undesired drug effects.

### BACKGROUND OF THE INVENTION

The present invention concerns a method for the analysis of cytosine methylations in DNA. 5-Methylcytosine is the most frequent covalently modified base in the DNA of eukaryotic cells. 5-Methylcytosine occurs only in the sequence context of CG dinucleotides, wherein, as a rule, the cytosines in both DNA strands are methylated. The specific methylation patterns of the DNA are also maintained during a DNA replication. In this way, first two hemimethylated DNA double strands are formed, which are subsequently converted to the fully methylated form. This conversion is produced by means of specific “maintenance” methyltransferases, e.g., DMNT1. These enzymes recognize specifically hemimethylated CG positions and methylate these by means of a methyl group donor, most commonly, S-adenosyl-L-methionine. The reaction mechanisms of maintenance methyltransferases have been described in detail (see, e.g., for DNMT1, Pradhan et al.: Recombinant human DNA (cytosine-5) methyltransferase. I. Expression, purification, and comparison of de novo and maintenance methylation. J Biol Chem. 1999 Nov. 12; 274 (46): 33002-10).

Cytosine methylation plays an important biological role, among other things, in the regulation of transcription, in genetic imprinting and in tumorigenesis (for review: Millar et al.: Five not four: History and significance of the fifth base. In: S. Beck and A. Olek, eds.: The Epigenome. Wiley-VCH Publishers Weinheim 2003, pp. 3-20). The identification of 5-methylcytosine as a component of genetic information is thus of considerable interest. A detection of methylation is difficult, of course, since cytosine and 5-methylcytosine have the same base-pairing behavior. Many of the conventional detection methods based on hybridization thus cannot distinguish between cytosine and methylcytosine. In addition, information of methylation is completely lost in a PCR amplification.

The conventional methods for methylation analysis operate essentially according to two different principles. In the first one, methylation-specific restriction enzymes are used, and in the second one, there occurs a selective chemical conversion of unmethylated cytosines to uracil (so-called bisulfite treatment, see, e.g.: DE 101 54 317 A1; DE 100 29 915 A1). The DNA that has been pretreated enzymatically or chemically is then usually amplified and can be analyzed in different ways (for review: WO 02/072880 p. 1 ff; Fraga and Esteller: DNA Methylation: A Profile of Methods and Applications. Biotechniques 33: 632-649, September 2002).

Due to the participation of cytosine methylation in the development of disease, particularly in tumorigenesis, diagnostic applications of methylation analysis are of great interest. Methods which permit detection of aberrant methylation patterns in body fluids, e.g., in serum, play a special role. Unlike unstable RNA, DNA is often encountered in body fluids. The DNA concentration in blood in fact is increased in destructive pathological processes such as cancer disorders. A diagnosis of cancer by means of a methylation analysis of tumor DNA found in body fluids is thus possible and has in fact been described many times (see e.g.: Palmisano et al.: Predicting lung cancer by detecting aberrant promoter methylation in sputum. Cancer Res. 2000 Nov. 1; 60(21): 5954-8). A difficulty here, however, consists of the fact that in body fluids, in addition to the DNA with the methylation pattern typical of disease, there is also found a large quantity of DNA of identical sequence, but of another methylation pattern. Diagnostic methods therefore are faced with the problem that they must be able to detect small quantities of specifically methylated DNA against an intense background of DNA of the same sequence but of a different methylation pattern. The applicability of this method has thus been limited up to now.

A way has now been found to increase the quantity of methylated DNA prior to analysis. In this way, a more sensitive detection of cytosine methylations and thus also an earlier diagnosis of disease are made possible.

The method according to the invention operates according to the following principle: A small quantity of specifically methylated DNA is found in the specimen under investigation. In addition, a large quantity of background DNA is present, in which the corresponding cytosine positions are present unmethylated. The DNA double strands are separated and then combined again. In this way, hybrid molecules are formed from methylated and unmethylated DNA. These hybrids serve as the substrate for a maintenance methyltransferase. The hemimethylated positions are thus converted into fully methylated positions. The quantity of methylated DNA is then doubled in the optimal case. By repeating the method, the proportion of methylated DNA can be further increased.

A similar method for the analysis of cytosine methylations is disclosed in Patent Application DE 102 14 232. Described therein is a method for a methylation-maintaining PCR. Also therein, the methylated DNA to be investigated is converted to hemimethylated DNA, which is then converted into fully methylated DNA by means of maintenance methyltransferases. However, the hemimethylated DNA in DE 102 14 232 is formed by extension of a primer hybridized to the DNA to be investigated. The method according to the present invention, in contrast, utilizes only the DNA present in the specimen for the hybrid formation. The use of primers is thus not necessary.

### DESCRIPTION

The method according to the invention makes possible a sensitive detection of methylated DNA against a background of unmethylated DNA. The method according to the invention is conducted by the following steps:
a) the double strands of the DNA to be investigated are separated and then reassociated with the formation of hemimethylated double strands,
b) the hemimethylated positions that are formed in step a) are converted into fully methylated positions by means of an enzyme,
c) the methylated DNA is analyzed.

The terms methylated, unmethylated, hemimethylated, and fully methylated thus do not describe the overall methylation state of the DNA, but only the state of the individual CpG positions within the DNA to be investigated. Methylated and fully methylated describe synonymously the case in which the positions to be investigated are methylated in both DNA strands. Background DNA is understood in the following as unmethylated DNA, which makes available the same base sequence as the methylated DNA to be investigated.

In the specimen to be investigated, the methylated DNA must be present against a background of unmethylated DNA. It is thus assured that after the separation and reassociation of the DNA, hemimethylated double strands are formed, which then can be converted into fully methylated DNA. The quantity of background DNA is preferred to be at least a factor of 20 higher, and particularly preferred, at least a factor of 50 higher than the quantity of methylated DNA. The DNA to be investigated can originate from different sources depending on the diagnostic or scientific objective. For diagnostic investigations, body fluids, in particular, can serve as the initial material, since in addition to the methylated DNA to be detected, there is a large background of unmethylated DNA present in such fluids. Serum is preferably used. It is, however, also possible to use DNA from sputum, stool, urine, or cerebrospinal fluid. Preferably, the DNA is isolated from biological specimens. The DNA is extracted according to standard methods, from blood, e.g., with the use of the Qiagen UltraSens DNA extraction kit. Other methods for DNA isolation are known to the person skilled in the art.

In a preferred embodiment, fragmented DNA is used. The separation and reassociation of the DNA strands can be facilitated in this way. A fragment length of 0.2 to 8 kB is preferred in this case. The fragmentation can be conducted, e.g., by reaction with restriction enzymes. The reaction conditions and the enzymes that can be employed are part of the prior art and are taken, e.g., from the protocols supplied by the manufacturers. Other methods for DNA fragmentation are known to the person skilled in the art. Particularly in plasma specimens, the DNA is already present in fragmented form. Another fragmentation is not necessary here.

The DNA is separated and reassociated preferably by changes in temperature. The use of other techniques for producing single-stranded DNA or for combining the single strands is also equally conceivable.

The enzymatic conversion of hemimethylated into fully methylated DNA preferably takes place with the use of a maintenance methyltransferase and a methyl group donor, e.g., S-adenosylmethionine. DNMT1 is preferably used as the enzyme. The reaction conditions of DNMT1 are part of the prior art and are provided, e.g., from the protocols of commercial vendors. It is known to the person skilled in the art that other enzymes that can convert hemimethylated positions into fully methylated positions can also be used.

In the optimal case, the quantity of methylated DNA can be doubled by separation, reassociation and enzyme conversion. By repeating this cycle, another increase in the proportion of methylated DNA can be achieved. How often these cycles can be conducted in a meaningful way depends on the ratio between methylated DNA and background DNA. An optimal number of cycles is easily determined experimentally.

In the case of repeated cycles, there is concern that a thermal separation of the double strands can lead to a denaturation of the methyltransferase. If this occurs, the enzyme must be added anew in each cycle. If, however, a heat-stable enzyme variant is available, then a repeated addition is not necessary.

In the last step of the method according to the invention, the methylated DNA is analyzed. A number of methods are known to the person skilled in the art for this purpose (for review: WO 02/072880 p. 1 ff; Fraga and Esteller, loc. cit.). Preferably, the DNA is first converted with a bisulfite reagent, which converts unmethylated cytosine into uracil, but leaves 5-methylcytosine unchanged (see, e.g.: DE 101 54 317 A1; DE 100 29 915 A1). A corresponding conversion is also conceivable with the use of methylation-specific cytidine deaminases (see: Bransteitter et al.: Activation-induced cytidine deaminase deaminates deoxycytidine on single-stranded DNA but requires the action of RNase. Proc Natl Acad Sci USA. 2003 Apr. 1; 100 (7): 4102-7).

The converted DNA can be analyzed in different ways. It is particularly preferred to amplify the DNA first by means of a polymerase chain reaction. Thus, a selective amplification of the methylated DNA can be assured via different methods, e.g., via the so-called “heavy methyl” method (for review: WO 02/072880) or the so-called “methylation-sensitive PCR” (“MSP”; see: Herman et al.: Methylation-specific PCR: a novel PCR assay for methylation status of CpG islands. Proc Natl Acad Sci USA. 1996 Sep. 3; 93 (18): 9821-6). The amplificates can be detected via conventional methods, e.g., via primer extension reactions (“MsSNuPE”; see, e.g,: DE 100 10 280) or via hybridization to oligomer arrays (see, e.g.: Adorjan et al., Tumour class prediction and discovery by microarray-based DNA methylation analysis. Nucleic Acids Res. 2002 Mar. 1; 30 (5): e2 1). In another particularly preferred embodiment, the amplificates are analyzed with the use of PCR real-time variants (see: U.S. Pat. No. 6,331,393 “Methyl-Light”). Preferred variants are therefore the “Taqman” and the “LightCycler” methods).

Another aspect of the invention consists of the use of all embodiments according to the invention. If disease-specific cytosine positions are investigated, then the method according to the invention is particularly suitable for the diagnosis or prognosis of cancer disorders or other diseases associated with a change of methylation status. These include, among others, CNS malfunctions; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disease of the skin, the muscles, the connective tissue or the bones; endocrine and metabolic malfunction, damage or disease; headaches or sexual malfunction. The method according to the invention is also suitable for predicting undesired drug effects, for establishing a specific drug therapy (personalized medicine) and for monitoring the success of a drug therapy. Another application is distinguishing cell types or tissues and investigating cell differentiation.

The person skilled in the art recognizes that the method according to the invention can also be performed in the reverse direction, as long as enzymes are available which specifically convert hemimethylated DNA into unmethylated DNA. A small quantity of unmethylated DNA can be detected herewith against a high background of methylated DNA. The double strands of the DNA to be investigated, as described above, are first separated and then reassociated with the formation of hemimethylated double strands. The DNA is then reacted with an enzyme that specifically removes the methyl groups at the hemimethylated positions. The quantity of unmethylated DNA thus can be increased. The further analysis can be conducted as described above.

### EXAMPLE OF EMBODIMENT

Identification of methylated GSTP1-exon 1 DNA in the plasma of prostate tumor patients.

The DNA was isolated from 1 ml of plasma with the QIAamp UltraSens Virus Kit (Qiagen, Hilden) according to the manufacturer's instructions. 50 μl of the isolated DNA (approximately 100 pg) were incubated for 10 min at 96° C. and then cooled within 60 min to 25° C. The DNA solution was then incubated with 2 units of human DNA-(cytosine-5)-methyltransferase (Dnmt1) of New England Biolabs according to the manufacturer's instructions for 2 h at 37° C. After this, a repeated incubation was carried out for 10 min at 96° C. Then the reaction solution was again cooled to 25° C. within 60 min, and, after the addition of 2 units of Dnmt1, was incubated for another 2 h at 37° C. Subsequently, the DNA solution was subjected to a bisulfite treatment (Olek et al. Nucleic Acids Res. 1996 Dec. 15; 24 (24): 5064-6). By this reaction, unmethylated cytosines are converted to uracils, while in contrast, methylated cytosines remain unchanged.

The methylated GSTP1-exon1 DNA fragments were then detected by a heavy methyl real-time PCR. For this purpose, the GSTp1-exon1 fragment (nt 1183 to nt 1303 in Genbank Accession M24485.1) was amplified in a reaction volume of 20 μl in einem LightCycler device (Roche Diagnostics). The real-time PCR reaction mix consisted of 10 μl of DNA, 2 μl of FastStart LightCycler reaction mix for hybridization probes (Roche Diagnostics, Penzberg), 0.30 μmol/l primer (SEQ ID NO: 1; GGGAttAtttTTATAAGGtT), 0.30 μmol/l primer (SEQ ID NO: 2; TaCTaaaAaCTCTaAaCCCCATC), 0.15 μmol/l fluorescein detection probe (SEQ ID NO: 3; TTCGtCGtCGtAGTtTTCGtt-fluorescein; TIB-MolBiol, Berlin), 0.15 μmol/l detection probe (SEQ ID NO: 4; red640-tAGTGAGTACGCGCGGtt-phosphate; TIB-MolBiol, Berlin), 4 μmol/l blocker oligonucleotide (SEQ ID NO: 5 CCCATCCCCaAAAACaCaAACCaCa-phosphate, TIB-MolBiol, Berlin) and 3.5 mmol/l MgCl₂. In the oligonucleotide sequences, those positions which corresponded tothe converted, originally unmethylated cytosines were designated with a lower-case “t” (or lower-case “a” in the complementary strand). In contrast, the capital “T” (or “A” in the complementary strand) stands for thymine that was already present prior to the bisulfite treatment.

The PCR conditions were as follows: an incubation for 10 min at 95° C., then 55 cycles with the following steps: 95° C. for 10 s, 56° C. for 30 s, and 72° C. for 10 s. The fluorescence was measured after the annealing phase at 56° C. in each cycle.

Comparative GSTp1 PCRs of Dmnt1-treated and untreated specimens showed that methylated GSTP1 DNA fragments could be detected 0.5 to 1.5 cycles earlier in Dmnt1-treated specimens. This corresponds to an increase in the methylated DNA of 50-150%.

1) A method for the detection of methylated DNA against a background of unmethylated DNA is hereby characterized in that
a) the double strands of the DNA to be investigated are separated and then reassociated with the formation of hemimethylated double strands,
b) the hemimethylated positions that are formed in step a) are converted into fully methylated positions by means of an enzyme,
c) the methylated DNA is analyzed.

2) The method according to claim 1, further characterized in that DNA from body fluids is investigated.

3) The method according to claim 2, further characterized in that DNA from serum is investigated.

4) The method according to claim 1, further characterized in that the DNA of step a) is fragmented.

5) The method according to claim 1, further characterized in that a maintenance methyltransferase is used in step b).

6) The method according to claim 5, further characterized in that DNMT1 is used as the maintenance methyltransferase.

7) The method according to claim 1, further characterized in that steps a) and b) are repeated once more or several times.

8) The method according to claim 1, further characterized in that a heat-stable methyltransferase is used.

9) The method according to claim 1, further characterized in that the DNA in step c) is first converted by means of a bisulfite reagent or enzymatically.

10) The method according to claim 9, further characterized in that the converted DNA is analyzed by means of one of the following methods: MSP, heavy methyl, MsSNuPE, methyl light.

11) Use of the method of any of claims 1-10 for the diagnosis or prognosis of cancer disorders or other diseases associated with a change in the cytosine methylation status, for predicting undesired drug effects, for establishing a specific drug therapy, for monitoring the success of a drug therapy, for distinguishing cell types or tissues and for investigating cell differentiation.

12) A method for the detection of unmethylated DNA against a background of methylated DNA is hereby characterized in that
a) the double strands of the DNA to be investigated are separated and then reassociated with the formation of hemimethylated double strands,
b) the hemimethylated positions that are formed in step a) are converted into unmethylated positions by means of an enzyme,
c) the unmethylated DNA is analyzed.
